(19) United States
(12) Patent Application Publication
(10) Pub. No.: US 2017/0151334 A1
(43) Pub. Date: Jun. 1, 2017

(54) INJECTABLE AND IMPLANTABLE CARRIER SYSTEMS BASED ON MODIFIED POLY(DICARBOXYLIC ACID MULTI-OIL ESTERS) FOR THE CONTROLLED RELEASE OF ACTIVE INGREDIENT

(71) Applicant: Martin-Luther-Universitaet Halle-Wittenberg, Halle (Saale) (DE)

(72) Inventors: Karsten MAEDER, Leipzig (DE); Verena WEISS, Glienecke (DE); Joerg KRESSLER, Halle (Saale) (DE); Toufik NAOLOU, Berlin (DE)

(21) Appl. No.: 15/305,595

(22) PCT Filed: Apr. 22, 2015
(86) PCT No.: PCT/DE2015/000204

§ 371 Date: Feb. 3, 2017

(30) Foreign Application Priority Data
Apr. 23, 2014 (DE) 10 2014 005 782.8

(51) Int. Cl. A61K 47/34; C08G 63/91; A61K 9/00; A61K 31/573; A61K 31/4422

(52) U.S. Cl. CPC A61K 47/34; A61K 31/573; A61K 31/4422; A61K 9/0019; A61K 9/0024; C08G 63/914

(57) ABSTRACT

Modified poly(dicarboxylic acid multiol esters) are suitable as carriers for bioactive substances, especially as injectable implants, without further additives. The polymers can be directly injected without using an organic solvent. Furthermore, modified poly(dicarboxylic acid multiol esters) can be mixed with suitable biocompatible organic solvents and injected or realized as implants. The carriers are suitable for controlled active ingredient release in human and veterinary medicine.

### BACKGROUND OF THE INVENTION

Carrier systems that release active ingredients in the body of animals or humans in a controlled manner are of great importance. Many highly effective active ingredients are characterized by a low bioavailability after oral administration and by a short half life due to a rapid excretion (elimination) or conversion (metabolism) of the active ingredient. Therapeutic use of such active ingredients is only possible with suitable active ingredient carrier systems, which are injected parenterally, and release the active ingredients in a time-controlled manner. The time period during which the active ingredient is released is dependent on the indications. A release of a few hours, days, weeks or several months may be desirable. Typical administration types are subcutaneous, intraperitoneal or intramuscular administration. In addition, these release systems are also used, for example, for local therapy in the brain, the eyes, ears, bone marrow and other organs. Depending on the indications, different release profiles can be desirable, such as continuous or pulsatile. The active ingredient carrier system must ensure reproducible, controlled release of the active ingredient and exhibit inert behavior with respect to the body.

At present, uses include both clinically biodegradable and non-degradable carrier materials. Non-biodegradable materials have the disadvantage that they must be removed from the body again. Examples include the products Implanon® (Firma Organon) and Vantas® (Orion Pharma), which must be removed from the body after 3 years and 12 months, respectively, and thus require a surgical procedure. Polylactic acid and copolymers of lactic acid and glycolic acid are primarily used as degradable materials. The monomers of these polymers are alpha-hydroxy acids. One disadvantage of these polymers is the complexity of the polymer degradation process. This is based on an autocatalytic hydrolysis, which can result in release kinetics that are difficult to control and very acid pH values (pH value of 2) /1, 2, 3/. The disadvantage is that the acidic microenvironment can cause active ingredients to be decomposed or inactivated even before they are released /4, 5/. The majority of products on the market are either microparticles or preformed implants (2014 Red List, Rote Liste® Service GmbH).

Microparticles, also referred to as microcapsules or microspherules, are solid, usually spherical particles having a size typically in the lower micrometer range. Examples of biodegradable microparticles are Enantone® (Takeda), Sixantone® (Takeda), Decapeptyl® (Ferring), Sandostatin® LAR®—(Sandoz), Pamorelin® LA 3.75 mg (Ipsen Pharma), Uropeptyl® Depot (Uropharm), Risperdal® Consta® (Janssen-Cilag). The production of microparticles having a narrow and reproducible size distribution is very demanding and complex. Primarily, coacervation, spray drying or emulsion techniques including solvent removal are employed /6/. Furthermore, the microparticles must be mixed with the dispersing agent prior to administration, which is time-consuming and often problematic given the poor wettability of the particles. Incomplete discharge from the syringe and clogging of the cannula constitute additional problems in the administration of microparticles. The problem of residue of organic, and usually chlorinated, solvents from the manufacturing process, as well as complex production under aseptic conditions and the difficulty of producing it on a large scale limit the possibilities of this pharmaceutical form.

Examples of preformed solid implants are the market products Zoladex® (AstraZeneca), Profact Depot (Sanofi), Leuprone® HEXAL® (Hexal), Leupro-Sandoz® (Sandoz), Ozurdex® (Allergan Pharmaceuticals). The problem with preformed solid implants is often that a relatively large cannula must be used to introduce the implant into the body, which moreover is often perceived as a foreign object and can cause pain.

More recent developments describe what are known as “in situ implants.” With these, a low-viscosity liquid is injected into the body which, in the body, transforms into a depot (such as a gel or a solid) by way of a stimulus (trigger). The stimulus used can be a solvent exchange, thermogelling, or a chemical reaction (cross-linking), for example /7/. Cross-linking reactions are difficult to control in vivo, and toxicity problems frequently occur with the cross-linking substances. The most important representatives of thermogelling systems include poloxamer-based systems, chitosan-containing preparations, and low molecular weight PEGylated polylactides or PEGylated poly(lactide-co-glycolides). The disadvantages of the above-mentioned thermogelling systems are potential gelling prior to administration, problems in terms of the stability of the excipients, excessively rapid release, and a lack of reproducibility of the release. In terms of systems based on solvent exchange, in contrast to other in-situ systems, commercial products are available. One typical example is Eligard®, which is sold in Germany by Astellas. N-methylpyrrolidone (NMP) is used as the solvent, in which the matrix-forming polymer is dissolved. In the body, the comingling of endogenous water and NMP results in reduced solubility and causes the polymer to precipitate. The disadvantages of this system are the inherent problems of autocatalytic polymer degradation found with polylactides and poly(lactide-co-glycolides), which often cause difficult-to-control and fluctuating release rates.

The use of injectable hydroxy ester-based polymers is described in WO 2007/012979. The disadvantage, however, is that covalent binding of the active ingredient is possible only to a very limited degree, or to an insufficient degree, since only the terminal groups of the polymer can be modified. In addition, the lipophilicity of the polymers described in the patent WO 2007/012979 can only be controlled to a very limited degree. The use of biodegradable carrier materials having a lipophilicity that can be matched to the active ingredient would therefore be a desirable objective.

As an alternative to polymer-based systems, the literature also describes lipid-based approaches as parenteral depot forms. Active ingredient-containing shaped bodies are produced by compressing or extruding solid lipids. The disadvantages of lipid systems based on glycerol fatty acid esters (monoglycerides, diglycerides or triglycerides) or waxes are the limited controllability of the lipophilicity and the occurrence of multiple polymorphic forms. The complex polymorphic behavior influences both the reproducibility of the active ingredient loading and active ingredient release, and the degradability of the active ingredient carriers. The patent DE 000003780862 describes solid compressed lipid products, which are administered by way of implantation. The patent WO 2009/080275 describes solid lipid implants produced by way of extrusion. Moreover, the production of compressed lipid products and extruded lipid products is described in the patent WO 2005/102284.

### SUMMARY OF THE INVENTION

It is the object of the invention to provide an alternative to the presently clinically used biodegradable monomer-based polymers having a hydroxycarboxylic acid structure. The problem was solved according to the invention by linear polyesters, which are created by esterifying dicarboxylic acids and polyhydric alcohols (diols, triols or higher alcohols) (FIG. 1).

If substances comprising three or a larger number of hydroxyl groups are used as the alcohol component, free hydroxyl groups are available in the polymer backbone following the polymerization when an appropriate synthesis strategy is employed. Enzymatic reactions using divinyl-activated dicarboxylic acids are preferred in this process, since these yield defined linear polymers having defined free hydroxyl groups and cross-linking is avoided /8/.

The free hydroxyl groups can be covalently chemically reacted either with active ingredients or with further excipients (FIG. 1).

So far, little is known about the covalent binding with active ingredients. Shafioul et al. published on the chemical attachment of the active ingredient xanthorrhizol to poly(dicarboxylic acid-multiol esters) and the enzymatic release thereof /9/. In contrast, esterification of poly(dicarboxylic acid multiol esters) with fatty acids has been described multiple times /10-16/, wherein the use of fatty acid-modified poly(dicarboxylic acid multiol esters) is presented in vitro for the production of active ingredient-containing microparticles and nanoparticles. Microparticles and nanoparticles, however, are complex to produce, and the particle size can often be difficult to control.

According to the invention, first it was found that modified poly(dicarboxylic acid multiol esters) are suitable as carriers for bioactive substances, especially injectable implants, without further additives. The polymers can be directly injected without using an organic solvent. The carriers of the invention are suitable for human and veterinary medicine.

Furthermore, it was secondly found that modified poly(dicarboxylic acid multiol esters) can be mixed with suitable biocompatible organic solvents and injected.

According to the invention, thirdly, modified poly(dicarboxylic acid multiol esters) can be realized as implants.

The present invention is characterized, more particularly, by the following.

According to the invention, there are provided or implantable carrier systems based on modified aliphatic polyesters which are, more particularly, modified poly(dicarboxylic acid multiol esters), for administration in humans or animals with the goal of the controlled release of a bioactive substance for therapeutic and/or diagnostic purposes, composed of polymers, the basic structure of which is characterized by a comb-like composition of the structural elements (A), (B) and (C),

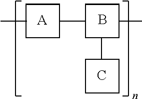

wherein the structural elements (A) and (B), which are composed of aliphatic dicarboxylic acids (A) and of polyols (B), form the polymer backbone, wherein two hydroxyl groups of the polyol (B) are esterified with the dicarboxylic acids of the structural element (A) to form a linear polymer backbone, and after the formation of the polymer backbone, which is composed of the structural elements (A) and (B) and linked by ester bonds, using triols and higher polyols in the structural element (B), one (in the case of triols) or more (number of hydroxyl groups in the polyol >3) free hydroxyl groups are still present, which completely or partially covalently or non-covalently bonded with aliphatic fatty acids and/or bioactive substances (structural element (C)).

The aliphatic dicarboxylic acid structural element (A) may be ethaneodoic acid or have the following general structure

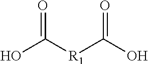

or
wherein a moiety R1 of 1 to 40 carbon atoms is between the two carboxyl groups, which moiety is a saturated or unsaturated, straight-chain or branched, aliphatic hydrocarbon moiety. Preferred are aliphatic dicarboxylic acids in which R1 comprises 1 to 14 carbon atoms, and, most preferably 4 to 10 carbon atoms.

The polyol structural element (B) may be composed of a mixture of diols, triols and higher polyols, i.e., a mixture of polyol.

The polyol structural unit is preferably composed of at least one diol having a chain length of 2 to 20 carbon atoms and, more preferably, at least one of ethylene glycol, propylene glycol, butane diol, hexane diol, octane diol and dodecane diol.

Structural element (B) may be composed 1 to 100%, preferably 3 to 40%, more preferably 3 to 10%, by mass, of polyols having at least three OH groups.

Preferred polyols having at least three hydroxyl groups are glycerol, erythritol, xylitol, mannitol, sorbitol, lactose, sucrose, trehalose, glucose, maltose, isomalt and maltitol.

The polyol (B) may be covalently bonded to further substances by way of the structural element (C), so that a comb-like polymer structure is created, wherein the degree of bonding can be between 0.1 and 100% of the free hydroxyl groups that are still present after the polymer backbone has been formed.

The structural element (C) may be a carboxylic acid, i.e., a fatty acid, or one or more bioactive substances, which is attached to the polyol structure (B) by way of an ester bond, wherein the fatty acid can be saturated or unsaturated, and the degree of esterification can range between 0 (zero) and 99.9% of the free hydroxyl groups.

The number of carbons in the carboxylic acid may be between 2 and 22, in particular between 8 and 22 carbon atoms, such as in caprylic acid (octanoic acid), capric acid (decanoic acid), lauric acid (dodecanoic acid), myristic acid (tetradecanoic acid), palmitic acid (hexadecanoic acid), stearic acid (octadecanoic acid), oleic acid ((9Z)-octadec-9-enoic acid), linoleic acid ((9Z,12Z)-octadeca-9,12-dienoic acid), and linolenic acid ((9Z,12Z,15Z)-octadeca-9,12,15-trienoic acid).

The bioactive substance(s) may be incorporated (not covalently or otherwise chemically bonded) or be present in a covalently bonded manner, wherein in the case of an incorporation (non-covalent bond) the bioactive substance(s) may be present in a dissolved, emulsified or suspended manner, and in the case of a covalent bond the bioactive substance(s) may be covalently bonded by an ester bond to the structural element B by way of a chemical reaction of a free hydroxyl group present in functional groups of the bioactive substance(s), such as an acid group.

The bioactive substances may have a low, average or high molecular weight, may be hydrophilic, amphiphilic or hydrophobic nature and present in dissolved, emulsified or suspended form.

The that bioactive substances may be one or more of antibiotics, anti-infective drugs, cytostatic drugs or other antineoplastic agents and protective drugs, local anesthetics, analgesics, neuropharmaceuticals, hormones, immunomodulators, immunosuppressants, antimycotics, anti-inflammatory agents, anti-hemorrhagic agents, antifibrinolytics, vitamins, hemostatic agents, antiphlogistics, beta receptor and calcium channel blockers, corticoids, withdrawal agents/agents for treating addictions, fibrinolytic drugs, antihypoxic drugs, cardiovascular drugs, anticoagulants, antiparasitic agents, anticaries, anti-periodontic agents, ophthalmic drugs, otologics, sexual hormones and the inhibitors thereof, Parkinson's drugs and other agents against extrapyramidal disorders, psychopharmaceutical drugs, serums, immunoglobulins, vaccinations, minerals, thrombocyte aggregation inhibitors, turberculosis drugs, diuretics, gastrointestinal drugs, weight loss drugs, analeptics, anti-rheumatism drugs, anti-allergy drugs, antianemic drugs, antiarrhythmic drugs, antidementia drugs, antidiabetic drugs, antidotes, antiemetics, antivertigo drugs, antiepilepstic drugs, antihypertensive drugs, antihypoglemic drugs, antihypotonics, antitussives, expectorants, atherosclerosis drugs, inhibitors of the renin-angiotensin-aldosterone system, broncholytics, antiasthmatic drugs and other agents for the respiratory tract, cholinergic drugs, antiseptics, diagnostic agents and agents for diagnostic preparation, diuretics, enzyme inhibitors, preparations for enzyme deficiency and transport proteins, geriatric drugs, gout drugs, gynecological drugs, hepatic drugs, hypnotic drugs, sedatives, pituitary gland hormones, hypothalamus hormones, regulatory peptides and analogs as well as the inhibitors thereof, cardiac drugs, coronary agents, lipid-lowering drugs, migraine drugs, muscle relaxers and reversal agents, anesthetics, neuropathy products and other neurotropic agents, osteoporosis agents/calcium regulators/bone metabolism regulators, thyroid therapy drugs, spasmolytics and anticholinergics, alternative agents, urological drugs and agents for treating hyperkalemia and hyperphosphatemia, vein therapy drugs, wound and scar treatment agents.

The active ingredient-containing carrier systems may be administered by way of implantation or an injection, such as subcutaneously, intraperitonealy and/or intramuscularly, as well as in or on the eyes, subarachinoidally, spinally, intracochlearly, intravestibularly (ear), intratympanically (extracochlearly) or intravenously for the targeted embolization of tumor-supplying blood vessels.

The carrier systems may be excipients such as:
a. water (in the concentration range from 0 to 90 percent (m/m), i.e., by mass;
b. excipients for stabilizing the active ingredient suspensions or emulsions, such as polyoxyethylene glycol polyhydroxy stearate, sorbitan fatty acid esters, polyoxyethylene sorbitan fatty acid esters, polyoxyethylene fatty acid esters, sugar esters, Pluronics (trademark for block copolymers of ethylene oxide and propylene oxide used as surfactants, wetting agents, emulsifiers and the like), lecithin, fatty acids, fatty alcohols, sterols, polyglyceryl fatty acid esters, triglycerides, and partial glycerides;
c. antioxidants (such as BHT, vitamin E, VIT E acetate);
d. viscosity-influencing substances; and
e. biocompatible organic solvents such as ethanol, dimethylsulfoxide, benzyl alcohol, benzyl benzoate, triacetin, n-methyl-2-pyrrolidone, polyethylene glycol, ethyl acrylate, glycofurol, 2-pyrrolidone, glycerol, n-propanol, triethyl citrate, propylene glycol, dimethyl acetate amide or mixtures of these, acetone, butanol, ethyl formate, acetic acid, pentanol, isopropanol, tetrahydrofuran, methyl acetate, ethyl lactate, propylene carbonate, and oleic acid.

The concentration of modified poly(dicarboxylic acid multiol esters) is 0.1% to 99.9999% (m/m), i.e., by mass.

### BRIEF DESCRIPTION OF THE DRAWINGS

FIG. 1:

Schematic illustration of basic structure of modified poly(dicarboxylic acid multiol esters)

Structural element (A): dicarboxylic acid

Structural element (B): polyol (diol, triol or higher polyol)

Structural element (C): covalently bonded active ingredient or excipient

The biodegradable polymer backbone is the result of the esterification of dicarboxylic acids (structural element (A)) and polyols (structural element (B)). Two hydroxyl groups of the polyol are esterified. In the case of higher polyols (number of hydroxyl groups greater than or equal to three), the hydroxyl groups that are still free after the polymer backbone has been formed can be partially or completely covalently bonded with bioactive substances or excipients (such as fatty acids) (structural element (C)).

FIG. 2:

Structural examples of fatty acid-modified poly(glycerol adipate). The polymer backbone is composed of poly(glycerol adipate). A portion of the hydroxyl groups still free after the polymer backbone has been formed is esterified with fatty acids (lauric acid, stearic acid, behenic acid, oleic acid).

FIG. 3:

Schematic illustration of the behavior of lauryl-substituted poly(glycerol adipate) polymers following an in-vitro injection with a 25-gauge needle into phosphate-buffered saline solution (PBS buffer, pH 7.4, diluted 1:10 with double distilled water). After one minute (b) and 6 hours (c) after injection (a). The implant drops to the bottom following injection and forms a spherical-like shape.

FIG. 4:

In-vitro time dependence of the fluorescent intensity of lauryl-substituted poly(glycerol adipic acid) polymers loaded with 1,1′-dioctadecyl-3,3,3′,3′-tetramethylindocarbocyanine perchlorate (DII). The model substance is released in vitro in a controlled manner over several weeks.

### DETAILED DESCRIPTION OF THE INVENTION

### Exemplary Embodiments

### Example 1

Fatty acid-modified poly(glycerol adipates) can be synthesized using the following or similar methods /10-16/.

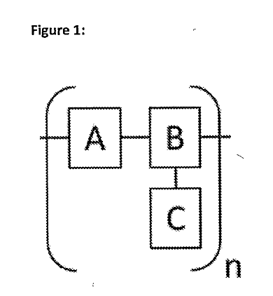

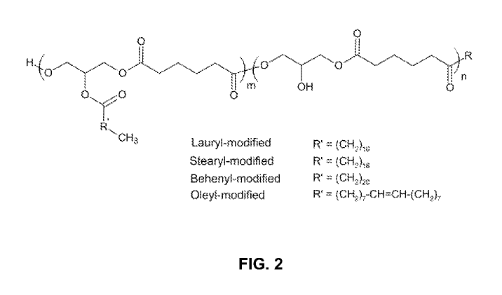

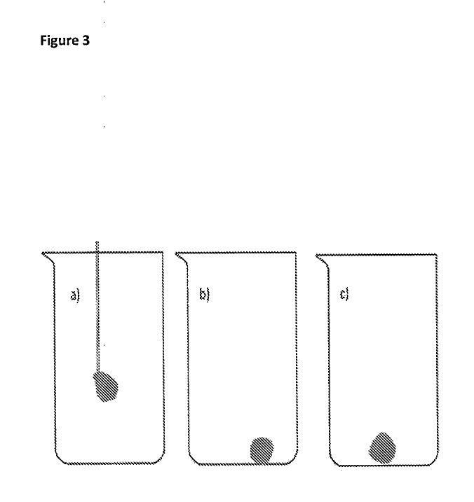

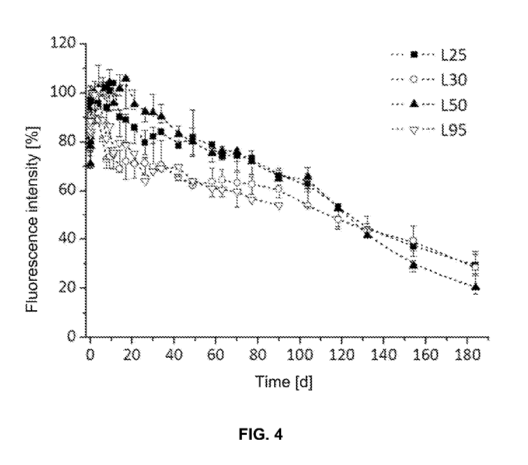

The structural element (A) (dicarboxylic acid) used is adipic acid, and glycerol, which comprises three hydroxyl groups, serves as the polyol (structural element (B)) (FIG. 1). Polyglycerol adipate, which is the polymer backbone, is realized, for example, by way of an enzymatic reaction of divinyl adipate or dimethyl adipate with glycerol in tetrahydrofuran or another suitable solvent /10/. Lauric acid, stearic acid, behenic acid or oleic acid are fatty acids used by way of example. The degree of substitution of the fatty acids ranges between 1% and 100% (based on the hydroxyl groups in the poly(glycerol adipate)) (FIG. 2).

The oleic acid- and lauric acid-modified polymers have a liquid consistency at a temperature of 20° C. (room temperature). The stearic acid- and behenic acid-modified polymers have a solid consistency at 20° C. They melt in the range of 30° C. to 45° C. (stearyl-modified poly(glyercol adipate)) and between 50° C. and 65° C. (behenyl-modified poly(glycerol adipate)), respectively.

In the case of the behenyl- and stearyl-modified polymers, an implantable shaped body can be obtained very easily by way of melt or extrusion processes.

### Example 2

Lauric acid-modified poly(glycerol adipate) (degree of esterification 25% based on free hydroxyl groups of the polyglycerol adipate) is injected through a 25-gauge needle in phosphate-buffered saline solution (PBS, diluted 1:10). A spherical depot forms (FIG. 3). (Thus, if a bioactive substance is included, a depot dosage is provided, a depot dosage being defined in medicine as a form of medication that can be stored in the patient's body for prolonged release.)

### Example 3

Lauryl acid-modified poly(glycerol adipate) (degree of esterification 95% based on free hydroxyl groups of the polyglycerol adipate) is injected through a 25-gauge needle in phosphate-buffered saline solution (PBS, diluted 1:10). A spherical depot forms.

### Example 4

Oleic acid-modified poly(glycerol adipate) (degree of esterification 15% based on free hydroxyl groups of the polyglycerol adipate) is injected through a 25-gauge needle in phosphate-buffered saline solution (PBS, diluted 1:10). A spherical depot forms.

### Example 5

Oleic acid-modified poly(glycerol adipate) (degree of esterification 92% based on free hydroxyl groups of the polyglycerol adipate) is injected through a 25-gauge needle in phosphate-buffered saline solution (PBS, diluted 1:10). A spherical depot forms.

### Example 6

Fatty acid-modified poly(glycerol sebacate) polymers are produced analogously to the synthesis pathways published in /10/. The structural element (A) (dicarboxylic acid) used is sebacic acid, and glycerol, which comprises three hydroxyl groups, serves as the polyol (structural element (B)) (FIG. 1). Poly(glycerol sebacate), which is the polymer backbone, is realized, for example, by way of an enzymatic reaction of divinyl sebacate with glycerol in tetrahydrofuran or another suitable solvent /10/. Lauric acid, stearic acid, behenic acid or oleic acid are fatty acids used by way of example. The degree of substitution of the fatty acids ranges between 1% and 100% (based on the hydroxyl groups of the poly(glycerol sebacate).

### Example 7

### Stearyl-Modified Poly(Sorbitol Adipate)

The polymer backbone is produced from sorbitol, serving as the polyol, and adipic acid, serving as the dicarboxylic acid, analogously to the guidelines published for poly(glycerol adipate) and is modified with stearic acid. At a degree of esterification of 30% (30% degree of esterification based on free hydroxyl groups of the poly(sorbitol adipate)), a polymer that is solid at room temperature and melts at 43° C. is created.

An implantable shaped body can be obtained very easily by way of melt or extrusion processes.

### Example 8

In-vitro release of the fluorescent dye 1,1′-dioctadecyl-3,3,3′,3′-tetramethylindocarbocyanine perchlorate (DII): lauryl-substituted poly(glycerol adipate) polymers were loaded with the DII fluorescent dye (1% m/m), and 60 microliters were incubated at 37° C. in vitro in PBS.

The following curves in FIG. 4 show a controlled release of the model substance in vitro. The number behind the L symbolizes the degree of substitution of lauric acid based on free hydroxyl groups of the poly(glycerol adipate).

### Example 9

In-vivo release of the fluorescent dye DII from lauryl-substituted poly(glycerol adipate)

The polymers were loaded with the fluorescent dye 1,1′-dioctadecyl-3,3,3′,3′-tetramethylindocarbocyanine perchlorate (DII) (0.5 to 1% m/m), and 50 microliters were subcutaneously injected into SKH1 mice.

In-vivo fluorescence images of the DII-loaded lauryl-modified poly(glycerol adipate) polymers following the subcutaneous injection (50 microliters) in SHK1 hairless mice proved the in-vivo release through visible signals. The degree of substitution of the lauric acid was 95% and 25%, respectively.

The results show a controlled in-vivo release over several weeks to months. Surprisingly, it was found that the more lipophilic implant (degree of substitution 95% based on free hydroxyl groups) is released more rapidly than the implant having a low substitution degree (25% based on free hydroxyl groups). The results demonstrate that the release of substances in vivo can be controlled by way of the degree of substitution with the fatty acid.

### Example 10

Production of liquid dexamethasone-containing implants. Dexamethasone is incorporated into fatty acid-modified poly(glycerol adipate) at a concentration of 10% m/m. The incorporation can take place at room temperature for the liquid polymers (lauryl- and oleyl-modified poly(glycerol adipate)). The dexamethasone-loaded (lauryl- and oleyl-modified poly(glycerol adipate) polymers can be injected, given the liquid consistency thereof.

### Example 11

Production of solid dexamethasone-containing implants. Dexamethasone is incorporated into fatty acid-modified poly(glycerol adipate) at a concentration of 20% m/m. The polymers that are solid at room temperature (such as stearyl-modified and behenyl-modified poly(glycerol adipate)) are melted, and the active ingredient is homogeneously incorporated. In the case of the solid polymers, a suitably shaped body (such as platelets or small rods), which can be implanted, can be obtained by way of melting and solidification, compression or extrusion.

### Example 12

Nifedipine is incorporated into stearyl-modified poly(sorbitol adipate) at a concentration of 10% m/m. For this purpose, the polymer is melted at 50° C., and the active ingredient is homogeneously distributed under protection against light. The melted active ingredient-containing polymer is brought into the desired size and shape by solidification in a suitable mold.

### Example 13

Active ingredient-containing in-situ implant. Stearyl-modified poly(glycerol adipate) is mixed with N-methylpyrrolidone (NMP) in a ratio of 1:1 (m/m). Leuprorelin acetate is homogeneously incorporated at a concentration of 5% (m/m). The preparation can be administered through a 25-gauge cannula, and forms an implant in situ after contact with water or in the interior of the body.

### Example 14

Implant comprising covalently bonded betulinic acid. The polymer backbone made of poly(glycerol adipate) is synthesized according to the guidelines described in the literature /10/. Instead of the fatty acids described in the literature /10/, the bioactive substance betulinic acid is covalently attached to the polymer backbone in an analogous reaction. The resultant betulinic acid poly(glycerol adipate) can either be converted into a desired implant form by melting or be applied as an in-situ implant with the aid of a biocompatible organic solvent (such as NMP).

### LIST OF REFERENCES

/1/ Mader et al., Biomaterials, 17(4), 457-461 (1996)
/2/ K Mader, G Bacic, A Domb, O Elmalak, R Langer and HM Swartz. J Pharm Sci 86: 126, (1997).
/3/ Liu and Schwendeman, Molecular Pharmaceutics, 9(5), 1342-1350(2012)
/4/ A Lucke, J Kiermaier and A Göpferich. Pharm Res 19: 175, (2002).
/5/ A Lucke and A Göpferich. Eur J Pharm Biopharm 55: 27, (2003).
/6/ Beck-Broichsitter et al., Parenterale Depotarzneiformen-Mikropartikel (Parenteral Depot Pharmaceutical Form Microparticles), in: Innovative Arzneiformen (Innovative Pharmaceutical forms), published by Mader and Weidenauer, Wissenschaftliche Verlagsgesellschaft Stuttgart, pgs. 235-262, (2010)
/7/ S Kempe and K Mader. J Control Release 161: 668, (2012)
/8/ B J Kline, E J Beckman, A J Russell. J Am Chem Soc 120: 9475 (1998)
/9/ Shafioul, Azam Sharif Mohammed; Pyo, Jung In; Kim, Kwan Soo; Cheong, Chan Seong: Synthesis of poly (glycerol-co-dioate-co-butanedioate-co-xanthorrhizol) ester and a study of chain length effect on pendant group loading. Journal of Molecular Catalysis B: Enzymatic (2012), 84, 198-204., DOI:10.1016/j.molcatb.2012.05.022
/10/ Naolou, T.; Weiss, V. M.; Conrad, D.; Busse, K.; Maeder, K.; Kressler, J.: Fatty acid modified poly(glycerol adipate)—polymeric analogues of glycerides. ACS Symposium Series(2013), 1135 (Tailored Polymer Architectures for Pharmaceutical and Biomedical Applications), 39-52, 14 pp., DOI:10.1021/bk-2013-1135.ch004
/11/ Kressler, Joerg; Naolou, Toufik; Conrad, Delf; Busse, Karsten; Amado, Elkin; Weiss, Verena; Maeder, Karsten: Fatty acid modified poly(glycerol adipate)-polymeric analogues of glycerides. Polymer Preprints (American Chemical Society, Division of Polymer Chemistry) (2012), 53(1), 567-568.
/12/ Weiss, Verena M.; Naolou, Toufik; Amado, Elkin; Busse, Karsten; Maeder, Karsten; Kressler, Joerg Formation of Structured Polygonal Nanoparticles by Phase-Separated Comb-Like Polymers. Macromolecular Rapid Communications(2012), 33(1), 35-40., DOI:10.1002/marc.201100565
/13/ Orafai, Hossein; Kallinteri, Paraskevi; Garnett, Martin; Huggins, Sean; Hutcheon, Gillian; Pourcain, Cristopher Novel poly(glycerol-adipate) polymers used for nanoparticle making: a study of surface free energy. Iranian Journal of Pharmaceutical Research (2008), 7(1), 11-19
/14/ Puri, Sanyogita; Kallinteri, Paraskevi; Higgins, Sean; Hutcheon, Gillian A.; Garnett, Martin C.: Drug incorporation and release of water soluble drugs from novel functionalized poly(glycerol adipate) nanoparticles. Journal of Controlled Release (2008), 125(1), 59-67
/15/ Kallinteri, Paraskevi; Higgins, Sean; Hutcheon, Gillian A.; St. Pourcain, Christopher B.; Garnett, Martin C.: Novel Functionalized Biodegradable Polymers for Nanoparticle Drug Delivery Systems. Biomacromolecules (2005), 6(4), 1885-1894
/16/ WO 2004096178 A1 20041111—Garnett, Martin Charles; Hutcheon, Gillian; Higgins, Sean; Kallinteri, Paraskevi; St. Pourcain, Chris: Nano and microparticle drug delivery systems comprising polyesters containing aliphatic dicarboxylate residues and residues of aliphatic polyols

1. An injectable or implantable carrier system comprising a carrier containing a bioactive substance for administration in humans or animals with for controlled release of the bioactive substance carried therein for a therapeutic and/or diagnostic purpose, of the carrier comprising a polymer of comb-like configuration of structural elements (A), (B) and (C), 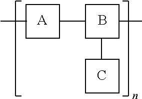
wherein the structural elements (A) and (B), which are comprised of an aliphatic dicarboxylic acid (A) and of a polyol (B) having at least three hydroxyl groups wherein two hydroxyl groups of the polyol (B) are esterified with the dicarboxylic acid of the structural element (A) to form a linear polymer backbone which is comprised of the structural elements (A) and (B) and linked by ester bonds and free hydroxyl groups on the polymer backbone completely or partially covalently or non-covalently bonded with an aliphatic and/or the bioactive substance (structural element (C)).

2. The carrier system according to claim 1, wherein the aliphatic dicarboxylic acid of the structural element (A) is ethanedoic acid or has the following general structure (I) 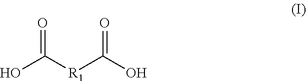
wherein R₁ is a moiety of 1-40 carbon atoms between the two carboxyl groups, which moiety is a saturated or unsaturated, straight-chain or branched aliphatic hydrocarbon moiety.

3. The carrier system according to claim 1, wherein aliphatic dicarboxylic acid has general structure (I) and the moiety R₁ has 1 to 14 carbon atoms.

4. The carrier system according to claim 1, wherein the polyol structural element (B) is further comprised of a diol.

5. The carrier system according to claim 1, wherein the diol has 2 to 20 carbon atoms.

6. The carrier system according to claims 1 and 4, characterized in that the proportion of higher polyols having at least 3 OH groups is between 1 and 100 percent.

7. The carrier system according to claim 1, wherein the polyol having at least three OH groups is glycerol, erythritol, xylitol, mannitol, sorbitol, lactose, sucrose, trehalose, glucose, maltose, isomalt or maltitol.

8. The carrier system according to claim 1, wherein between 0.1 and 100% of the free hydroxyl groups the polymer backbone are covalently bonded with an aliphatic fatty acid or a bioactive substance.

9. The carrier system according to claim 1, wherein the fatty acid is saturated or unsaturated, and the degree of esterification is between 0 and 99.9% of the free hydroxyl groups.

10. The carrier system according to claim 1, wherein the number of carbon atoms in the carboxylic acid is between 2 and 22.

11. The carrier system according to claim 1, wherein the bioactive substance is contained in the carrier as a solution, emulsion or suspension but not chemically bonded to the carrier or is covalently bonded to the carrier by an ester bond to the structural element B by way of a chemical reaction of a free hydroxyl group in the carrier.

12. The carrier system according to claim 11, wherein the bioactive substance has a low, average or high molecular weight, is hydrophilic, amphiphilic or hydrophobic nature and is contained in the carrier as a solution, emulsion or suspension.

13. The carrier system according to claim 11, wherein the bioactive substance is selected from the group consisting of antibiotics, anti-infective drugs, cytostatic drugs, other antineoplastic agents, protective drugs, local anesthetics, analgesics, neuropharmaceuticals, hormones, immunomodulators, immunosuppressants, antimycotics, anti-inflammatory agents, analgesics, anti-hemorrhagic agents, antifibrinolytics, vitamins, hemostatic agents, antiphlogistics, beta receptor and calcium channel blockers, corticoids, withdrawal agents/agents for treating addictions, fibrinolytic drugs, antihypoxic drugs, cardiovascular drugs, anticoagulants, antiparasitic agents, anticaries, anti-periodontic agents, ophthalmic drugs, otologics, sexual hormones and the inhibitors thereof, Parkinson's drugs and other agents against extrapyramidal disorders, psychopharmaceutical drugs, serums, immunoglobulins, vaccinations, minerals, thrombocyte aggregation inhibitors, turberculosis drugs, diuretics, gastrointestinal drugs, weight loss drugs, analeptics, anti-rheumatism drugs, anti-allergy drugs, antianemic drugs, antiarrhythmic drugs, antidementia drugs, antidiabetic drugs, antidotes, antiemetics, antivertigo drugs, antiepilepstic drugs, antihypertensive drugs, antihypoglemic drugs, antihypotonics, antitussives, expectorants, atherosclerosis drugs, inhibitors of the renin-angiotensin-aldosterone system, broncholytics, antiasthmatic drugs and other agents for the respiratory tract, cholinergic drugs, antiseptics, diagnostic agents and agents for diagnostic preparation, diuretics, enzyme inhibitors, preparations for enzyme deficiency and transport proteins, geriatric drugs, gout drugs, gynecological drugs, hepatic drugs, hypnotic drugs, sedatives, pituitary gland hormones, hypothalamus hormones, regulatory peptides and analogs as well as the inhibitors thereof, immunomodulators, cardiac drugs, coronary agents, lipid-lowering drugs, local anesthetics, gastrointestinal drugs, migraine drugs, muscle relaxers and reversal agents, anesthetics, neuropathy products and other neurotropic agents, osteoporosis agents/calcium regulators/bone metabolism regulators, thyroid therapy drugs, spasmolytics and anticholinergics, alternative agents, urological drugs and agents for treating hyperkalemia and hyperphosphatemia, vein therapy drugs, wound and scar treatment agents.

14. The carrier system according to claim 1, wherein the carrier system is injectable or implantable subcutaneously, intraperitonealy and/or intramuscularly, as well as in or on the eyes, subarachinoidally, spinally, intracochlearly, intravestibularly (ear), intratympanically (extracochlearly) or intravenously for the targeted embolization of tumor-supplying blood vessels.

15. The carrier system according to claim 1, further composing at least one of the following excipients:
a. water in the concentration range from 0 to 90 percent of the mass of the carrier system;
b. excipients for stabilizing suspensions or emulsions of the bioactive substance;
c. antioxidants;
d. viscosity-influencing substances; and
e. biocompatible organic solvents.

16. The carrier system according to claim 1, wherein the concentration of the polymer is 0.1% to 99.9999% of the mass of the carrier system.

17. A method of administering a bioactive substance to a human or animal, comprising injecting into the human or animal a carrier the bioactive carrier system according to claim 11 is carried.

18. The carrier system according to claim 3, wherein the moiety R₁ has 4 to 10 carbon atoms.

19. The carrier system according to claim 5, wherein the diol is ethylene glycol, propylene glycol, butane diol, hexane diol, octane diol or dodecane diol.

20. The carrier system according to claim 1, wherein 1 to 100%, by mass, of the polyol has at least three OH groups.

21. The carrier system according to claim 1, wherein the 3 to 40%, by mass, of the polyol has at least three OH groups.

22. The carrier system according to claim 1, wherein 3 to 10%, by mass, of the polyol has at least three OH groups.

23. The carrier system according to claim 10, wherein the number of carbon atoms in the carboxylic acid is between 2 and 22.

24. The carrier system according to claim 10, wherein the carboxylic acid is caprylic acid, capric acid, is lauric acid, myristic acid, palmitic acid, stearic acid, oleic acid, linoleic acid, or linolenic acid.

25. The carrier system according to claim 15, wherein the excipients for stabilizing suspensions or emulsions of the bioactive substance are selected from the group consisting of polyoxyethylene glycol poly hydroxy stearate, sorbitan fatty acid esters, polyoxyethylene sorbitan fatty acid esters, polyoxyethylene fatty acid esters, sugar esters, pluronics, lecithin, fatty acids, fatty alcohols, sterols, polyglyceryl fatty acid esters, triglycerides, and partial glycerides, the antioxidants are selected from the group consisting of BHT, vitamin E and vitamin E acetate, and the biocompatible organic solvents are selected from the group consisting of ethanol, dimethyl sulfoxide, benzyl alcohol, benzyl benzoate, triccetin, n-methyl-2-pyrrolidone, polyethylene glycol, ethyl acrylate, glycofurol, 2-pyrrolidone, glycerol, n-propanol, triethyl citrate, propylene glycol, dimethyl acetate amide, acetone, butanol, ethyl formate, acetic acid, pentanol, isopropanol, tetrahydrofuran, methyl acetate, ethyl lactate, propylene carbonate, and oleic acid.
